(19) United States
(12) Patent Application Publication
(10) Pub. No.: US 2005/0065091 A1
(43) Pub. Date: Mar. 24, 2005

(54) Stabilized ocular solutions

(76) Inventor: Gholam Peyman, New Orleans, LA (US)

Correspondence Address: WOOD, HERRON & EVANS, LLP, 2700 CAREW TOWER, 441 VINE STREET, CINCINNATI, OH, 45202, US

(21) Appl. No.: 10/666,629

(22) Filed: Sep. 18, 2003

(51) Int. Cl. A61K 38/04; A61K 31/355; A61K 31/375; A61K 31/198

(52) U.S. Cl. 514/18; 514/458; 514/474; 514/562

(57) ABSTRACT

Ocular solutions containing an antioxidant provide beneficial properties, for example, the antioxidant scavenges free radicals in the solution which may cause the solution to deteriorate. However, antioxidants are themselves extremely susceptible to oxidation. A stabilizing agent for the antioxidant retards or prevents the antioxidant from undesirable reactions and thus enhances its ability to stabilize the ocular solution. This in turn enhances the physiological properties of the ocular solution, which may be a topical solution such as eye drops, or a surgical ocular irrigation or volume replacement solution.

### FIELD OF THE INVENTION

The invention is directed to ocular solutions containing antioxidant compositions which have been stabilized to retard their deterioration.

### BACKGROUND

The eye is naturally bathed internally and externally by ocular fluids. The external portion of the eye is lubricated by lacrimal fluids (tears). The internal portion of the eye has two fluid-containing chambers: the anterior chamber contains the aqueous humor or aqueous, and the posterior chamber contain the vitreous humor or vitreous.

Various conditions require the need to introduce fluids into or on the surface of the eye to replace or replenish naturally occurring fluids. The loss of naturally occurring ocular fluids may be due to normal aging, pathological conditions, surgical intervention, etc. For example, during ocular surgery, the vitreous is frequently removed and must thereafter be replaced. Commercially available irrigating solutions are often used to replace some or all of the vitreous, such as irrigating solutions infused to replace vitreous removed during vitrectomy and thereby to maintain the shape of the globe. The composition and other properties of these solutions may affect the surgical outcome for the patient, for example, a solution that affects the clarity of the cornea and lens may result in decreased visual acuity. Additionally, swelling of the cornea during vitrectomy may be influenced by components of the irrigating solution. Other conditions such as dry eye disease result in decreased external lubrication, and topical solutions such as eye drops are often used to provide relief. Eye wash solutions are used to remove foreign material from the eye.

Ocular solutions, for either topical application or introduction into the eye, should be physiologically compatible and should maintain the physiologic integrity of the eye. They should be easy to prepare and should be stable in composition. The invention describes such compositions and method of using the compositions.

### SUMMARY OF THE INVENTION

Ocular solutions containing an antioxidant provide beneficial properties, for example, the antioxidant scavenges free radicals in the solution which may cause the solution to deteriorate. However, antioxidants are themselves extremely susceptible to oxidation. A stabilizing agent for the antioxidant retards or prevents the antioxidant from undesirable reactions and thus enhances its ability to stabilize the ocular solution. This in turn enhances the physiological properties of the ocular solution, which may be a topical solution such as eye drops, or a surgical ocular irrigation or volume replacement solution.

One embodiment of the invention is a composition comprising an ocular solution containing Vitamin C or Vitamin E and at least one stabilizing agent in an amount effective to stabilize the solution against oxidation. The stabilizing agent may be cysteine, L-cystine, glutathione, L-methionine, and/or N-acetyl-L-cysteine. Vitamin C or Vitamin E may be in a concentration in the range of about 1 μg/ml to about 10 mg/ml.

The stabilizing agent may be a solution of up to about 12% water and at least one water miscible organic solvent such as N-propanol, isopropanol, methanol, propylene glycol, butylene glycol, hexylene glycol, glycerine, sorbitol (polyol), di-propylene glycol, polypropylene glycol, a mixture of propylene glycol and butylene glycol with propylene glycol at about 25% by weight to about 80% by weight and butylene glycol at about 5% by weight to about 30% by weight. The stabilizing agent may be magnesium ions in at least 14 parts by weight to 100 parts by weight of a vitamin antioxidant. The stabilizing agent may be at least one phosphonic acid derivative and at least one metabisulfite. The stabilizing agent may be acrylic and methacrylic polymers, or xanthans. The stabilizing agent may be an extract of the fruit of the Emblica officinalis plant.

The ocular solution may be formulated as a true solution, or may be a suspension, a cream, a gel, an emulsion, or an ointment; the term solution is intended to encompass the different formulations and physical states.

In one embodiment, Vitamin C or Vitamin E is in a nonaqueous or substantially anhydrous silicone vehicle that is at least 50% by weight of the composition.

Various concentrations of Vitamin C or Vitamin E are possible. For example, Vitamin C or Vitamin E may range from about 0.025 mg/ml to about 1.2 mg/ml; from about 0.1 mg/ml to about 0.3 mg/ml; up to about 10% of the ocular solution; in the range of about 10% of the ocular solution to about 15% of the ocular solution within the limits of solubility.

Where the stabilizing agent is free cysteine, it may be present at a concentration by weight of the antioxidant in the range of about 0.4%, about 0.5%, about 0.6%, about 0.7%, about 0.8%, about 0.9%, about 1%, about 2.5%, or about 5%.

In various embodiments, free cysteine may be present at a concentration, relative to Vitamin C and/or Vitamin E, from about 0.2% to about 2.3%, or from about 0.2% to about 1.25%, or from about 0.3% to about 0.9%.

A composition may contain Vitamin C and/or Vitamin E in the range between about 1% by weight to about 25% by weight, glutathione in the range between about 0.01% by weight to about 10% by weight, a source of selenium at a concentration in the range from about 0.001% by weight to about 2.0% by weight, and a sulfur-containing amino acid at a concentration in the range of about 0.001% by weight to about 2.0% by weight.

In another embodiment, a composition comprising a physiologically acceptable formulation of Vitamin C and at least one stabilizing agent capable of retarding Vitamin C deterioration for use in a physiologically acceptable ocular solution is disclosed. Vitamin C, also called ascorbic acid, may be present in the form of derivatives or salts, such as sodium ascorbate, potassium ascorbate, calcium ascorbate, magnesium ascorbate, ascorbyl palmitate ester, ascorbyl laureate ester, ascorbyl myristate ester, ascorbyl stearate ester, magnesium ascorbyl phosphate, ascorbyl-phosphoryl-cholesterol, dipalmitate ascorbate, and ascorbate anhydrides.

Vitamin C may be at a concentration in the range of about 1 μg/ml of the ocular solution to about 10 mg/ml of the ocular solution, or in the range of about 0.025 mg/ml of the ocular solution to about 1.2 mg/ml of the ocular solution, or in the range of about 0.1 mg/ml of the ocular solution to about 0.3 mg/ml of the ocular solution, or at a concentration up to about 10% of the ocular solution, or in the range of about 10% of the ocular solution to about 15% of the ocular solution within the limits of solubility, or at a concentration in the range of about 0.025 mg/ml of the ocular solution to about 1.2 mg/ml of the ocular solution. Free cysteine as the stabilizing agent may be present at a concentration by weight, relative to Vitamin C, ranging from about 0.2% to about 2.3%, or ranging from about 0.2% to about 1.25%, or ranging from about 0.3% to about 0.9%.

In one embodiment, the composition is Vitamin C in the range between about 1% by weight to about 25% by weight, glutathione in the range between about 0.01% by weight to about 10% by weight, a source of selenium at a concentration in the range from about 0.001% by weight to about 2.0% by weight, and a sulfur-containing amino acid at a concentration in the range of about 0.001% by weight to about 2.0% by weight.

A method for stabilizing an ocular solution is also disclosed where a stabilizing agent is provided to an ocular solution containing Vitamin C, with the amount of the stabilizing agent sufficient to retard oxidation of Vitamin C and thus stabilize the ocular solution. The ocular solution may be for external use, such as a topical lubricant, contact lens solution, or eye wash solution. The ocular solution may be for internal use, such as an irrigating solution or a volume replacement solution.

A method for stabilizing an ocular solution is also disclosed by providing a stabilizing agent to an ocular solution containing an antioxidant. The stabilizing agent may be cysteine, magnesium ions, magnesium sulfate heptahydrate, L-methionine, N-acetyl-L-cysteine, glutathione, a mixture of propylene glycol and butylene glycol, at least one phosphonic acid derivative and at least one metabisufite, a combination of polysilicone-11, dimethicone, and cyclomethicone; xanthan polymers; acrylic and methacrylic polymers; or an extract of the fruit of the Emblica officinalis plant. The amount of stabilizing agent added is effective to retard oxidation of the antioxidant.

These and other advantages will be apparent in light of the following figures and detailed description.

### DETAILED DESCRIPTION

An ocular solution containing an antioxidant that has been stabilized to retard deterioration of the antioxidant is disclosed. The ocular solution containing a stabilized antioxidant is able to participate in reactions that scavenge free radicals and thus preserve the physiological benefits of the antioxidant. In contrast, because antioxidants are inherently unstable and readily participate in free radical reactions during which they are auto-oxidized, an ocular solution containing an antioxidant that has not been stabilized is extremely susceptible to oxidation. This renders the antioxidant less active, and decreases, retards, or prevents the antioxidant from providing its beneficial properties to the ocular solution.

The ocular solution to which the stabilized antioxidant is added may be any physiologically compatible ocular solution for use in any manner, either topically or invasively. It will be appreciated that the ocular solution containing the stabilized antioxidant need not be in the physical form of a true solution, but instead may be a suspension, a cream, an ointment, an emulsion, a gel, etc. Thus, the term solution is used for convenience but encompasses other physical states in which the stabilized form of the antioxidant and the other components are present. It will also be appreciated that the stabilized antioxidant may be included in the formulation for preparing an ocular solution, or may be added in dry form or in the form of a concentrated solution to an already prepared ocular solution.

The ocular solution may be one that is used as an ocular irrigating solution or as a volume replacement during ocular surgery. It may also be one that is used topically, and thus encompasses eye drops, eye wash solutions, and contact lens solutions. It may be used in over the counter (OTC) ocular solutions for topical application, for example, in ocular solutions such as artificial tears or lubricants. One commercially available ophthalmic lubricant (Viva-Drops®, available from Vision Pharmaceuticals, Inc. (Mitchell S.D.)) is reported to contain polysorbate 80 as an antioxidant active ingredient, and sodium citrate, citric acid, EDTA, retinyl palmitate, and sodium pyruvate as inactive ingredients and antioxidants. It may also be used in prescription (Rx) ocular solutions for topical application. Examples of prescription ophthalmic compositions include, but are not limited to, the following: loteprednol etabonate ophthalmic suspension 0.5% (Lotemax™) as an ophthalmic topical antiinflammatory corticosterioid; loteprednol etabonate ophthalmic suspension 0.2% (Alrex™) as an ophthalmic topical anti-inflammatory cortocosteroid; metipranolol ophthalmic solution 0.3% (OptiPranolol™) as a non-selective beta-adrenergic receptor blocking agent; sodium chloride 2% or 5% solution or ointment (Muro 128R) as a treatment for corneal edema by drawing water out of the cornea of the eye, all available from Bausch and Lomb Pharmaceuticals (Tampa Fla.); and trifluridine ophthalmic solution 1% (ViropticR) available from Monarch Pharmaceuticals, Inc. (Bristol Tenn.).

The inventive composition may be used in physiologic ophthalmic irrigating solutions. One example is Balanced Salt Solution (BSS®, Alcon Laboratories (Randburg, South Africa), containing per ml 0.64% sodium chloride, 0.075% potassium chloride, 0.048% calcium chloride, 0.03% magnesium chloride, 0.39% sodium acetate, and 0.17% sodium citrate dihydrate, as well as sodium hydroxide and/or hydrochloric acid to adjust pH, and water for injection. Another example is Ocular Irrigation Solution® (Allergan, Irvine Calif.). Another example is lactated Ringer's solution. Another example is a normal saline solution. Another example is normal saline adjusted to pH 7.4 with sodium bicarbonate. The inventive composition may be used in ophthalmic volume replacement solutions for introduction into the posterior chamber of the eye to replace the vitreous that is removed during vitrectomy. As another example, it may be used as an ocular wash solution.

Any antioxidant in a physiological formulation for ocular administration may be used. One antioxidant is Vitamin C, which is also known as ascorbic acid or L-ascorbic acid. Vitamin C is unstable in the presence of oxygen and decomposes to form L-ascorbic acid 2-hydrogen sulfate, and then dehydroascorbic acid. Providing a stabilizing agent with Vitamin C reduces or eliminates its tendency to be oxidized in solution, and hence the stabilizing agent guards against Vitamin C deterioration. Another example of an antioxidant is Vitamin E (α-tocopherol). Vitamin E may be in the form of tocopherol or its esters, for example, tocopheryl acetate. Another example of an antioxidant is Vitamin A, which may be in the form of retinol or its ester or acids, for example, retinyl palmitate or retinoic acid. Thus, it will be appreciated that derivatives of Vitamins C, E, and A are also included within the scope antioxidants. A stabilized form of any of these antioxidants may be used separately or in combination in the ocular solution.

An antioxidant and a stabilizing agent for the antioxidant is included with ocular solutions for any use. The antioxidant and stabilizing agent may be added together or separately as individual components in the preparation of an ocular solution. Alternatively, a solution of the antioxidant and stabilizing agent may be prepared and then added to the ocular solution. It will be appreciated that, if the ocular solution to which the antioxidant is to be added itself contains a stabilizing agent, then the separate addition of a stabilizing agent may be optional and the antioxidant may be added directly to the solution to result in a stabilized antioxidant. For example, some commercially available ocular solutions contain glutathione, which is an antioxidant stabilizing agent. The solutions may be commercial irrigating solutions that contain other known components, such as various anions and cations, buffers to regulate pH, adenosine, calcium, glucose, bicarbonate, dextrose, dextran 40 (a low molecular weight colloidal osmotic agent), gentamicin, dexamethasone, selenium, zinc, and gluconide. The antioxidant and stabilizing agent may be added to commercial ocular lubricating solutions, such as artificial tears. The antioxidant and stabilizing agent may be added to commercial ocular wash solutions. Any solution for ocular administration, either administration to the exterior surface of the eye or to one of the interior chambers of the eye, may contain the antioxidant and stabilizer.

In one embodiment, a sterile solution of the antioxidant such as Vitamin C is prepared. A stabilized form of Vitamin C is prepared by including in the Vitamin C solution one or more components which inhibit, minimize, prevent, or decrease the extent of oxidation. One example of such a stabilizing component is cysteine. Another example of such a component is L-cystine. Another example is a solution of water up to about 12% water and at least one organic solvent miscible with water, namely, ethanol, N-propanol, isopropanol, methanol, propylene glycol, butylene glycol, hexylene glycol, glycerine, sorbitol (polyol), di-propylene glycol, polypropylene glycol (claim 1 of '382 patent), or a mixture of propylene glycol and butylene glycol with propylene glycol at about 25% by weight to about 80% by weight and butylene glycol at about 5% by weight to about 30% by weight and optionally including other glycols. Another example is glutathione, such as reduced glutathione with selenium as a cofactor. Another example is N-acetyl-L-cysteine. Another example is L-methionine. Another example is magnesium ions in at least 14 parts by weight to 100 parts by weight Vitamin C. Another example is a combination of at least one phosphonic acid derivative and at least one metabisulfite. Another example is an antioxidant in a nonaqueous or substantially anhydrous silicone vehicle where the silicone vehicle comprises at least 50% by weight of the composition. Another example is acrylic and methacrylic polymers, or xanthans. Another example is an extract of the fruit of the Emblica officinalis plant, which contains, by weight, gallic/ellagic acid derivatives of 2-keto-glucono-δ-lactone at about 35% to about 55%; Punigluconin (2,3-di-O-galloyl 4,6-(S)-hexahydroxy-diphenoylgluconic acid at about 4% to about 15%; Pedunculagin (2,3,4,6-bis-(S)-hexahydroxydiphenoyl-D-glucose at about 10% to about 20%; Rutin (flavanol-3)glycoside at about 5% to about 15%; low to medium molecular weight gallo-ellagi tannoids at about 10% to about 30%, gallic acid from 0% to about 5%, and ellagic acid from 0% to about 5%, available as CAPROS from Natreon Inc. (New Brunswick N.J.).

The antioxidant Vitamins C, A, and E are available commercially from a number of sources (e.g., Sigma-Aldrich Fine Chemicals, St. Louis Mo.). A solution of Vitamin C is prepared in a desired concentration. In one embodiment, the solvent is water. In another embodiment, the solvent is water and at least one organic liquid miscible with water. Vitamin C derivatives may also be used, example of which include alkali salts such as sodium ascorbate and potassium ascorbate, alkaline earth salts such as calcium ascorbate and magnesium ascorbate, esters such as ascorbyl palmitate, ascorbyl laureate, ascorbyl myristate, ascorbyl stearate, other salts such as magnesium ascorbyl phosphate, ascorbyl-phosphoryl-cholesterol, dipalmitate ascorbate, and ascorbate anhydrides.

The stabilizing agents are available commercially from a number of sources (e.g., Sigma-Aldrich). Cysteine and methionine are available as a hydrochloride salt or another physiologically acceptable salt, and may be added to the solution in amounts to yield an appropriate amount of the free base.

Embodiments of the invention include various concentrations of the antioxidants and stabilizer sufficient to stabilize the antioxidants against oxidation. Concentrations of the antioxidant and stabilizer(s) may depend upon the use for the composition, as is known to one skilled in the art. Thus, the invention is not limited to a specific concentration of either Vitamin C or the stabilizing agent. In general, the antioxidant is present in the ocular solution at concentrations ranging from about 1 μg/ml to about 10 mg/ml. In embodiments, Vitamin C, Vitamin A, or Vitamin E at concentrations in the range of about 0.025 mg/ml to about 1.2 mg/ml may be used, or concentrations in the range of about 0.1 mg/ml to 0.3 mg/ml may be used, or a concentration up to about 10% of the final solution may be used, or a concentration in the range of about 10% of the final solution to about 15% of the final solution within the limits of solubility may be used. Free cysteine is included at concentrations by weight of the vitamin of about 0.4%, about 0.5%, about 0.6%, about 0.7%, about 0.8%, about 0.9%, about 1%, about 2.5%, or about 5% may be used, or within the range of about 0.2% of the vitamin to about 2.3% of the vitamin, or within the range of about 0.2% of the vitamin to about 1.25% of the vitamin, or within the range of about 0.3% of the vitamin to about 0.9% of the vitamin. In one embodiment, an ocular solution contains Vitamin C, Vitamin A, or Vitamin E at a concentration in the range between about 1% by weight to about 25% by weight, glutathione in the range between about 0.01% by weight to about 10% by weight, a source of selenium as a cofactor for glutathione at a concentration in the range from about 0.001% by weight to about 2.0% by weight, and a sulfur-containing amino acid at a concentration in the range of about 0.001% by weight to about 2.0% by weight.

In various embodiments, other precautions may also be taken to minimize or reduce oxidization and thus further enhance the stability of the ocular solution. For example, the ocular solution containing antioxidant may contain a chelating agent such as ethylenediamine tetraacedic acid (EDTA), it may be packaged under nitrogen, its exposure to light may be minimized, etc.

The ocular solution may be an ocular wash solution, an ocular lubricating solution, an ocular irrigating solution, an ocular therapeutic solution, etc. The following references disclose methods which may be used in embodiments of the invention and are expressly incorporated by reference herein in their entirety: U.S. Pat. Nos. 3,958,017; 4,983,382; 5,281,196; 5,516,793; 5,703,122; 5,906,811; 6,804,110; 6,087,393; 6,103,267; 6,110,476; 6,146,664; 6,183,729; 6,211,231; 6,235,721; 6,299,889; 6,361,783. The following examples are illustrative only, and do not limit the scope of the invention.

### EXAMPLE 1

An aqueous solution of up to about 10% Vitamin C, containing in the range of about 1% cysteine to about 5% cysteine is prepared. The stabilized Vitamin C solution is prepared with or is incorporated into an ocular solution to achieve a final Vitamin C concentration of about 0.1% by weight to about 5% by weight.

### EXAMPLE 2

An aqueous solution containing in the range of about 0.025 mg/ml Vitamin C to about 1.2 mg/ml Vitamin C, and in the range of about 1% cysteine to about 5% cysteine, is prepared. The stabilized Vitamin C solution is prepared with or is incorporated into an ocular solution to achieve a final Vitamin C concentration of about 0.1% by weight to about 5% by weight.

### EXAMPLE 3

An aqueous solution containing about 0.228 mg/ml Vitamin C and in the range of about 1% cysteine to about 5% cysteine is prepared. The stabilized Vitamin C solution is prepared with or is incorporated into an ocular solution to achieve a final Vitamin C concentration of about 0.1% by weight to about 5% by weight.

### EXAMPLE 4

An aqueous solution containing about 0.1 mg/ml Vitamin C to about 0.3 mg/ml Vitamin C and in the range of about 1% cysteine to about 5% cysteine is prepared. The stabilized Vitamin C solution is prepared with or is incorporated into an ocular solution to achieve a final Vitamin C concentration of about 0.1% by weight to about 5.0% by weight.

### EXAMPLE 5

Vitamin C at about 0.78 mg/ml (23.04 mg/ounce) and free cysteine in the range of about 0.0097 mg/ml (about 0.288 mg/1 ounce) to about 0.019 mg/ml (about 0.576 mg/1 ounce) is prepared with or is incorporated into an ocular solution to achieve a final Vitamin C concentration of about 0.1% by weight to about 5% by weight.

### EXAMPLE 6

Vitamin C in the range between about 0.29 mg/ml to about 0.39 mg/ml, and cysteine hydrochloride anhydrous at a concentration of about 0.002 mg/ml as free cysteine, or in the range between about 0.505% to about 0.685% free cysteine by weight Vitamin C, is prepared with or is added to an ocular solution.

### EXAMPLE 7

An ocular solution that may be a contact lens solution, a eye wash solution, an irrigating solution, a volume replacement solution, a therapeutic solution available either by prescription or over the counter, or a lubricant solution contains about 0.0340% by weight Vitamin C and 0.0002% cysteine.

### EXAMPLE 8

An ocular solution containing up to about 10% Vitamin C and cysteine at a concentration in the range of about 0.2% by weight of Vitamin C to about 2.3% by weight of the Vitamin C is prepared.

### EXAMPLE 9

An ocular solution containing up to about 10% Vitamin C and cysteine at a concentration of about 0.588% by weight Vitamin C is prepared.

### EXAMPLE 10

An ocular solution containing in the range of about 0.0025% Vitamin C to about 0.12% Vitamin C, and cysteine at a concentration of about 0.588% by weight Vitamin C, is prepared.

### EXAMPLE 11

An ocular solution containing in the range of about 30 mg Vitamin C to about 2000 mg Vitamin C, and magnesium ions at least at 14 parts by weight in 100 parts of Vitamin C, is blended at concentrations in the range of about 1.5 mEq/liter to about 35 mEq/liter.

### EXAMPLE 12

In 50 mM phosphate buffer (pH 6), magnesium sulfate heptahydrate is dissolved at 2.054 g/liter, and Vitamin C at 0.2 g/liter. The solution is transferred into polyethylene bags, replaced with nitrogen, and sterilized under nitrogen pressure for 15 min at 115° C. It is added to or formulated with an ocular solution to achieve a final Vitamin C concentration in the range between about 0.1% by weight to about 5% by weight.

### EXAMPLE 13

To an ocular solution, the following components are added: 334 mg/liter Vitamin C, 4 g/liter L-methionine, 1.1 g/liter N-acetyl-L-cysteine, and 2.054 g/liter magnesium sulfate heptahydrate.

### EXAMPLE 14

To an ocular solution, at least 5% Vitamin C and a mixture of propylene glycol and butylene glycol, with propylene glycol at about 25% by weight to about 80% by weight and butylene glycol at about 5% by weight to about 30% by weight, and optionally including other glycols, is added

### EXAMPLE 15

To an ocular solution, at least 5% Vitamin C and a mixture of propylene glycol and butylene glycol, with propylene glycol at about 25% by weight to about 80% by weight and butylene glycol at about 5% by weight to about 30% by weight, and optionally including other glycols, is added

### EXAMPLE 16

Vitamin C is added to an ocular solution in an oil phase dispersion of particles. Vitamin C, water, and a water soluble or water dispersible polymer(s) is prepared. The polymers may be natural or synthetic polymers, including but not limited to methacrylates, cellulosic polymers, polyethylene glycols and copolymers, natural or modified natural resins, polyvinyl resins, water-solubilized or water-dispersible polyurethanes, water-solubilized or water-dispersible ethers, etc. A solution of Vitamin C (in various embodiments, by weight of the dispersion/suspension, at least 5%, at least 5.5%, at least 6%, at least 7%, at least 8.5%, at least 10%, and up to 40%, 50%, 60%, 75%), water, and a water-soluble polymer is prepared and mixed with a solution of oil and water in a surface active agent having an hydrophilic-lipophilic balance of less than 12. The solutions are dispersed to form a mixture, which is cooled to solidify the Vitamin C containing solution to form particles dispersed in oil. Emulsifying is performed at a temperature greater than 40° C.

### EXAMPLE 17

An ocular solution is prepared with Vitamin C at a concentration in the range of about 0.01% to about 20%, at least one phosphonic acid derivative at a concentration between about 0.005% to about 5%, and at least one metabisulfite in a concentration between about 0.005% to about 5%. The phosphonic acid derivative may be ethylenediaminetetra(methylenephosphonic acid), hexamethylenediaminetetra(methylenephosphonic acid), diethylenetriaminepenta(methylenephosphonic acid), and their salts. The metabisulfite may be an alkali-metal, alkaline-earth, metal, or ammonium salt of anhydrosulfonic acid. The weight ratio between metabisulfite and phonphonic acid derivative ranges from 1 to 1000. In one embodiment, the weight ratio ranges from 1 to 5.

### EXAMPLE 18

Vitamin C in a nonaqueous or substantially anhydrous silicone vehicle is prepared. In various embodiments, Vitamin C is at a concentration (all percentages are by weight) of at least 0.1%, at least 1%, from about 2% to about 30%, from about 5% to about 20%, from about 8% to about 12%, or about 40% of undissolved ascorbic acid. The carrier is an anydrous silicone carrier in an amount of about 50% by weight to about 80% by weight. The silicone vehicle may be a oil, gel, or solid. Silicone includes organosiloxanes and polyorganosiloxanes. Other antioxidants may be included.

Polysilicone-11 (from about 0.1% to about 68%), dimethicone (from about 0.1% to about 36%), and cyclomethicone (from about 0.1% to about 56%) are combined and the optional vitamins, if used, are added. Solid Vitamin C (10%) is dispersed with agitation and is ground using a three-roll mill until the particle size is less than 20 μm and the mixture is uniform. In one embodiment, the particle size is less than 12.5 μm.

### EXAMPLE 19

In various embodiments, Vitamin C is at a concentration ranging from about 5% to about 70%, from about 10% to about 60%, or from about 20% to about 60%. Xanthan or acrylic and methacrylic polymers are added to a concentration ranging from about 0.1% to about 5%. The composition is prepared with or is incorporated in an ocular solution at a Vitamin C concentration in the range between about 0.1% to about 5%. Linoleic acid or an ester of linoleic acid may also be included.

### EXAMPLE 20

In various embodiments, Vitamin C or derivatives of Vitamin C and an extract of the fruit of the Emblica officinalis plant are combined in a weight ratio of about 1:10 or about 10:1. The use of the fruit extract of Emblica officinalis as a stabilizer for Vitamin C is described in U.S. Pat. No. 6,235,721 which is expressly incorporated by reference herein in its entirety. The extract contains, by weight, (1) and (2) about 35-55% of the gallic/ellagic acid derivatives of 2-keto-glucono-δ-lactone; (3) about 4-15% of 2,3-di-O-galloyl 4,6-(S)-hexahydroxydiphenoyl-gluconic acid; (4) about 10-20% of 2,3,4,6-bis-(S)-hexahydroxydiphenoyl-D-glucose; (5) about 5-15% of 3′,4′,5,7-tetrahydroxyflavone-3-O-rhamnoglucoside; and (6) about 10-30% of tannoids of gallic/ellagic acid, gallic acid (0-5%); ellagic acid (0-5%) at a concentration ranging from about 5% to about 70%, from about 10% to about 60%, or from about 20% to about 60%. Xanthan or acrylic and methacrylic polymers are added at a concentration ranging from about 0.1% to about 5%. The composition is incorporated in an ocular solution at a Vitamin C concentration in the range between about 0.1% to about 5%.

Other variations or embodiments of the invention will also be apparent to one of ordinary skill in the art from the above figures and descriptions. Thus, the forgoing embodiments are not to be construed as limiting the scope of this invention.

1. A composition comprising an ocular solution containing Vitamin C or Vitamin E and at least one stabilizing agent in an amount effective to stabilize the solution against oxidation.

2. The composition of claim 1 wherein the stabilizing agent is selected from at least one of cysteine, L-cysteine, glutathione, L-methionine, and N-acetyl-L-cysteine.

3. The composition of claim 1 wherein Vitamin C or Vitamin E is in a concentration in the range of about 1 μg/ml to about 10 mg/ml.

4. The composition of claim 1 wherein the stabilizing agent comprises a solution of up to about 12% water and at least one water miscible organic solvent selected from the group consisting of N-propanol, isopropanol, methanol, propylene glycol, butylene glycol, hexylene glycol, glycerine, sorbitol (polyol), di-propylene glycol, polypropylene glycol, a mixture of propylene glycol and butylene glycol with propylene glycol at about 25% by weight to about 80% by weight and butylene glycol at about 5% by weight to about 30% by weight.

5. The composition of claim 1 wherein the stabilizing agent comprises magnesium ions in at least 14 parts by weight to 100 parts by weight of a vitamin antioxidant.

6. The composition of claim 1 wherein the stabilizing agent comprises a combination of at least one phosphonic acid derivative and at least one metabisulfite.

7. The composition of claim 1 wherein the stabilizing agent comprises at least one of acrylic and methacrylic polymers, or xanthans.

8. The composition of claim 1 wherein the stabilizing agent comprises an extract of the fruit of the Emblica officinalis plant.

9. The composition of claim 1 in a formulation selected from at least one of a suspension, a cream, a gel, an emulsion, an ointment, and a solution.

10. The composition of claim 1 wherein Vitamin C or Vitamin E is in a nonaqueous or substantially anhydrous silicone vehicle where the silicone vehicle comprises at least 50% by weight of the composition.

11. The composition of claim 1 comprising at least one of Vitamin C or Vitamin E at concentrations in the range of about 0.025 mg/ml to about 1.2 mg/ml.

12. The composition of claim 1 comprising at least one of Vitamin C or Vitamin E at concentrations in the range of about 0.1 mg/ml to about 0.3 mg/ml.

13. The composition of claim 1 comprising at least one of Vitamin C or Vitamin E at concentrations up to about 10% of the ocular solution.

14. The composition of claim 1 comprising at least one of Vitamin C or Vitamin E at a concentration in the range of about 10% of the ocular solution to about 15% of the ocular solution within the limits of solubility.

15. The composition of claim 1 wherein the stabilizing agent is free cysteine at a concentration by weight of the antioxidant in the range of about 0.4%, about 0.5%, about 0.6%, about 0.7%, about 0.8%, about 0.9%, about 1%, about 2.5%, or about 5%.

16. The composition of claim 1 wherein the stabilizing agent is free cysteine at a concentration, relative to at least one of Vitamin C or Vitamin E, in a range selected from at least one of about 0.2% to about 2.3%, about 0.2% to about 1.25%, or about 0.3% to about 0.9%.

17. The composition of claim 1 comprising at least one of Vitamin C or Vitamin E in the range between about 1% by weight to about 25% by weight, glutathione in the range between about 0.01% by weight to about 10% by weight, a source of selenium at a concentration in the range from about 0.001% by weight to about 2.0% by weight, and a sulfur-containing amino acid at a concentration in the range of about 0.001% by weight to about 2.0% by weight.

18. A composition comprising a physiologically acceptable formulation of Vitamin C and at least one stabilizing agent capable of retarding Vitamin C deterioration for use in a physiologically acceptable ocular solution.

19. The composition of claim 18 wherein the stabilizing agent is chosen from at least one of cysteine, L-cystine, glutathione, and L-methionine,

20. The composition of claim 18 wherein the stabilizing agent comprises a solution of up to about 12% water and at least one water miscible organic solvent selected from the group consisting of N-propanol, isopropanol, methanol, propylene glycol, butylene glycol, hexylene glycol, glycerine, sorbitol (polyol), di-propylene glycol, polypropylene glycol, a mixture of propylene glycol and butylene glycol with propylene glycol at about 25% by weight to about 80% by weight and butylene glycol at about 5% by weight to about 30% by weight.

21. The composition of claim 18 wherein Vitamin C is selected from at least one of sodium acerbate, potassium ascorbate, calcium ascorbate, magnesium ascorbate, ascorbyl palmitate ester, ascorbyl laureate ester, ascorbyl myristate ester, ascorbyl stearate ester, magnesium ascorbyl phosphate, ascorbyl-phosphoryl-cholesterol, dipalmitate ascorbate, and ascorbate anhydrides.

22. The composition of claim 18 comprising Vitamin C at a concentration in the range of about 1 μg/ml of the ocular solution to about 10 mg/ml of the ocular solution.

23. The-composition of claim 18 comprising Vitamin C at a concentration in the range of about 0.025 mg/ml of the ocular solution to about 1.2 mg/ml of the ocular solution.

24. The composition of claim 18 comprising Vitamin C at a concentration in the range of about 0.1 mg/ml of the ocular solution to about 0.3 mg/ml of the ocular solution.

25. The composition of claim 18 comprising Vitamin C at a concentration up to about 10% of the ocular solution.

26. The composition of claim 18 comprising Vitamin C at a concentration in the range of about 10% of the ocular solution to about 15% of the ocular solution within the limits of solubility.

27. The composition of claim 18 comprising Vitamin C at a concentration in the range of about 0.025 mg/ml of the ocular solution to about 1.2 mg/ml of the ocular solution.

28. The composition of claim 18 wherein the stabilizing agent is free cysteine is at a concentration by weight relative to Vitamin C in the range of about 0.4%, about 0.5%, about 0.6%, about 0.7%, about 0.8%, about 0.9%, about 1%, about 2.5%, or about 5%

29. The composition of claim 18 wherein the stabilizing agent is free cysteine at a concentration by weight relative to Vitamin C in the range selected from at least one of about 0.2% to about 2.3%, about 0.2% to about 1.25%, or about 0.3% to about 0.9%.

30. The composition of claim 18 comprising Vitamin C in the range between about 1% by weight to about 25% by weight, glutathione in the range between about 0.01% by weight to about 10% by weight, a source of selenium at a concentration in the range from about 0.001% by weight to about 2.0% by weight, and a sulfur-containing amino acid at a concentration in the range of about 0.001% by weight to about 2.0% by weight.

31. A method for stabilizing an ocular solution comprising providing a stabilizing agent to an ocular solution containing Vitamin C, the stabilizing agent in an amount sufficient to retard oxidation of Vitamin C and thus stabilize the ocular solution.

32. The method of claim 31 wherein the ocular solution is for external ocular application.

33. The method of claim 31 wherein the ocular solution is at least one of an ocular lubrication solution, a contact lens solution, and an ocular wash solution.

34. The method of claim 31 wherein the ocular solution is for internal ocular instillation.

35. The method of claim 31 wherein the ocular solution is for at least one of irrigation or volume replacement.

36. The method of claim 31 wherein the Vitamin C and the stabilizing agent are provided to an ocular solution.

37. The method of claim 31 wherein the stabilizing agent is provided to an ocular solution containing Vitamin C.

38. The method of claim 31 wherein the stabilizing agent provided is selected from at least one of cysteine, L-cystine, glutathione, L-methionine, and N-acetyl-L-cysteine.

39. The method of claim 31 wherein Vitamin C is at a concentration in the range of about 1 μg/ml to about 10 mg/ml.

40. The method of claim 31 wherein the stabilizing agent provided comprises a solution of up to about 12% water and at least one water miscible organic solvent selected from the group consisting of N-propanol, isopropanol, methanol, propylene glycol, butylene glycol, hexylene glycol, glycerine, sorbitol (polyol), di-propylene glycol, polypropylene glycol, a mixture of propylene glycol and butylene glycol with propylene glycol at about 25% by weight to about 80% by weight and butylene glycol at about 5% by weight to about 30% by weight.

41. The method of claim 31 wherein the stabilizing agent provided comprises magnesium ions in at least 14 parts by weight to 100 parts by weight of a vitamin antioxidant.

42. The method of claim 31 wherein the stabilizing agent provided comprises a combination of at least one phosphonic acid derivative and at least one metabisulfite.

43. The method of claim 31 wherein Vitamin C is in a nonaqueous or substantially anhydrous silicone vehicle where the silicone vehicle comprises at least 50% by weight of the composition.

44. The method of claim 31 wherein the stabilizing agent provided comprises at least one of acrylic and methacrylic polymers, or xanthans.

45. The method of claim 31 wherein the stabilizing agent provided comprises an extract of the fruit of the Emblica officinalis plant.

46. The method of claim 31 wherein the ocular solution is in a formulation selected from at least one of a suspension, a cream, a gel, an emulsion, an ointment, and a solution.

47. A method for stabilizing an ocular solution comprising providing to an ocular solution containing an antioxidant at least one stabilizing agent in an amount effective to stabilize the solution against oxidation, the stabilizing agent selected from at least one of cysteine, magnesium ions, magnesium sulfate heptahydrate, L-methionine, N-acetyl-L-cysteine, glutathione, a mixture of propylene glycol and butylene glycol, at least one phosphonic acid derivative and at least one metabisulfite, a combination of polysilicone-11, dimethicone, and cyclomethicone; xanthan polymers; acrylic and methacrylic polymers; and an extract of the fruit of the Emblica officinalis plant.

48. A composition comprising an ocular solution containing at least one stabilizing agent for an antioxidant, the stabilizing agent in an amount effective to stabilize the solution and selected from at least one of cysteine, magnesium ions, magnesium sulfate heptahydrate, L-methionine, N-acetyl-L-cysteine, glutathione, a mixture of propylene glycol and butylene glycol, at least one phosphonic acid derivative and at least one metabisulfite, a combination of polysilicone-11, dimethicone, and cyclomethicone; xanthan polymers; acrylic and methacrylic polymers; and an extract of the fruit of the Emblica officinalis plant.

49. An ocular composition comprising a physiologically acceptable formulation of Vitamin C in an amount to provide an antioxidant effect and at least one stabilizing agent in an amount effective to retard Vitamin C oxidation in a physiologically acceptable ocular solution.

50. A physiologically acceptable ocular solution comprising a balanced salt solution, a physiologically acceptable formulation of an antioxidant, and at least one stabilizing agent in an effective amount to retard oxidation of the antioxidant.

51. The solution of claim 49 wherein the stabilizing agent is at least one of cysteine, magnesium ions, magnesium sulfate heptahydrate, L-methionine, N-acetyl-L-cysteine, glutathione, a mixture of propylene glycol and butylene glycol, at least one phosphonic acid derivative and at least one metabisulfite, a combination of polysilicone-11, dimethicone, and cyclomethicone; xanthan polymers; acrylic and methacrylic polymers; or an extract of the fruit of the Emblica officinalis plant.

52. The solution of claim 50 wherein the stabilizing agent is glutathione.

53. A method of preparing an ocular solution comprising providing at least one antioxidant to a glutathione-containing ocular solution in an amount to provide an antioxidant effect.

54. The method of claim 53 wherein the antioxidant is at least one of Vitamin C, Vitamin E, or Vitamin A.

55. The method of claim 53 wherein the antioxidant is in the range of about 0.025 mg/ml to about 1.2 mg/ml.

56. The method of claim 53 wherein the antioxidant is up to about 10% of the ocular solution.

57. The method of claim 53 wherein the antioxidant is in the range of about 10% of the ocular solution to about 15% of the ocular solution within the limits of solubility.

58. The method of claim 53 wherein the antioxidant is in the range of about 1 μg/ml to about 10 mg/ml.

59. A method of preparing a stabilized ocular solution comprising adding to a physiologically acceptable ocular solution comprising per ml 0.64% sodium chloride, 0.075% potassium chloride, 0.048% calcium chloride, 0.03% magnesium chloride, 0.39% sodium acetate, and 0.17% sodium citrate dihydrate, and glutathione, an antioxidant in an amount effective to provide an antioxidant effect thereby forming a stabilized antioxidant ocular solution.

60. The solution of claim 59 wherein the antioxidant is at least one of Vitamin C, Vitamin E, Vitamin A, or derivatives thereof.

61. The solution of claim 59 wherein the concentration of antioxidant is in the range of about 0.025 mg/ml to about 1.2 mg/ml.

62. The solution of claim 59 wherein the concentration of antioxidant is up to about 10% of the ocular solution.

63. The solution of claim 59 wherein the concentration of antioxidant is in the range of about 10% of the ocular solution to about 15% of the ocular solution within the limits of solubility.

64. The solution of claim 59 wherein the concentration of antioxidant is in the range of about 1 μg/ml to about 10 mg/ml.
